(19) United States
(12) Patent Application Publication
(10) Pub. No.: US 2012/0259316 A1
(43) Pub. Date: Oct. 11, 2012

(54) CATHETER

(76) Inventor: Jiro Takashima, Houston, TX (US)

(21) Appl. No.: 13/446,632

(22) Filed: Apr. 13, 2012

Related U.S. Application Data
(63) Continuation-in-part of application No. 12/684,806, filed on Jan. 8, 2010, now abandoned.
(60) Provisional application No. 61/143,632, filed on Jan. 9, 2009.

(51) Int. Cl. A61M 25/00

(52) U.S. Cl. 604/523

(57) ABSTRACT

A catheter in accordance with one embodiment of the invention includes a first end, a second end, and a body, which comprises one or more segments, disposed between said first end and said second end, wherein at least one of the one or more segments has a first section and a second section, wherein the first section has a first tapered surface with a diameter increasing from a first edge to a ridge and a second section has a second tapered surface with a diameter decreasing from the ridge to a second edge, and wherein a first volume enclosed between said first tapered surface in the first section and an imaginary cylinder circumscribing the ridge is smaller than a second volume enclosed between the second tapered surface in the second section and the imaginary cylinder.

### CROSS-REFERENCE TO RELATED APPLICATIONS

This application is a continuation-in-part application of U.S. patent application Ser. No. 12/684,806, filed on Jan. 12, 2009, which claims the benefit, under 35 U.S.C. Section 119(e), of provisional patent application No. 61/143,632, filed on Jan. 9, 2009. This application claims the benefits of these prior filed applications and incorporates the disclosures of these prior filed applications by reference in their entireties.

### BACKGROUND OF INVENTION

1. Field of the Invention

The present invention relates generally to catheters. More particularly, the present invention relates to self-retaining catheters.

2. Background Art

A catheter is a tubular instrument used to allow fluid to pass from or into a body cavity. For example, catheters are commonly used to drain urine from the urinary bladder. However, catheters are also used for drug and intravenous fluid delivery, angioplasty, and in the case of a Swan-Ganz catheter, the direct measurement of blood pressure in a vein or artery. This is not an exhaustive list, and the various types of catheters are abundant as are their uses.

An issue commonly associated with catheters is retention failure. Retention failure occurs when the catheter fails to remain at the desired location, and this can lead to loss of catheter function and exacerbation of the underlying problem that necessitated a catheter, as well as other issues such as infection, contamination, and discomfort. When catheter retention failure occurs, reinsertion is a typical response. Reinsertion can increase the probability of infection and trauma to the body cavity. Recurring episodes of retention failures and the resulting reinsertions can diminish a patient's willingness to seek medical help for their underlying issues.

When a traditional cylindrical catheter is inserted into the human body cavity, the compression force created by peristaltic movement inside a human body cavity wall will gradually expel the catheter. Therefore, some catheters may provide a stop means (such as a balloon) to prevent the peristaltic movement within a body cavity from expelling the catheter.

One example of a catheter with a stop means is a balloon-tip catheter. The balloon, upon inflation inside a body cavity or lumen, acts to resist peristalsis, as well as other physiological factors that contribute to retention failure and catheter expulsion from a body cavity. An example of a balloon-tip catheter is the Foley catheter. However, certain issues can arise with the use of a Foley catheter. First, upon inflation, the balloon may rupture. This may require surgery to repair any internal damage, as well as to remove the ruptured balloon fragments. Second, the balloon may be inadvertently inflated before reaching the inflation destination. This can be very painful for the user and may require invasive techniques to withdraw the catheter.

Another potential solution to the catheter retention problem is to have “screw-type threads” on the exterior surface of the catheter. The threads can also facilitate the insertion of a catheter. That is, the catheter can be inserted into a body cavity by rotating the device. However, major problems and complications can arise if the catheter is removed improperly. For example, if the catheter is accidentally pulled out, the body cavity can suffer devastating injuries.

A third potential solution to retention failure problem is to include external protrusions on the exterior surface of a catheter. These protrusions can interact with the walls of the body cavity and act as anchors to prevent retention failure. However, the use of anchors can lead to irritation of the body cavity and user discomfort.

U.S. Pat. No. 5,964,732, issued to Willard, discloses methods of positioning a catheter within a urethra. This patent also describes that overcoming hydraulic forces acting to expel the catheter can be accomplished by compressive forces generated by the urethral wall acting on the longitudinal surface of the catheter. Willard also describes that a combination of surface projections with the longitudinal surface area may offset the hydraulic and physiological forces that act to expel the catheter from the urethra. Willard generally states that retention can be achieved if the sum of forces between the urethra and the body of the catheter exceed the hydraulic and physiological forces acting to expel the catheter.

U.S. Pat. No. 5,971,967, issued to Willard, describes a urethral catheter having one or more tapered anchors located on the external surface of the device. The anchors form partial spiral helices. Willard discloses this conformation overcomes deficiencies in the prior art, namely, that a continuous helical surface provides a shunt pathway for urine.

While these prior art approaches can overcome problems related to retention of catheters in body cavities, there remains a need for other catheters that would not have retention failure and are easy to manufacture.

### SUMMARY OF INVENTION

One aspect of the invention relates to catheters for use in a body cavity. A catheter in accordance with one embodiment of the invention includes a first end, a second end, and a body, which comprises one or more segments, disposed between said first end and said second end, wherein at least one of the one or more segments has a first section and a second section, wherein the first section has a first tapered surface with a diameter increasing from a first edge to a ridge and a second section has a second tapered surface with a diameter decreasing from the ridge to a second edge, and wherein a first volume enclosed between said first tapered surface in the first section and an imaginary cylinder circumscribing the ridge is smaller than a second volume enclosed between the second tapered surface in the second section and the imaginary cylinder.

In accordance with some embodiments of the invention, the second section is longer than the first section in a segment. In some embodiments, the first section has a convex profile and the second section has a concave profile. In some embodiments, both the first section and the second section have a convex profile.

Other aspects and advantages of the invention will be apparent from the following description and the appended claims.

### BRIEF DESCRIPTION OF DRAWINGS

FIG. 1 shows a cross sectional view of a catheter according to one embodiment of the present invention.

FIG. 2A shows an expanded view of a segment of the catheter shown in FIG. 1. FIG. 2B shows a schematic illustrating the forces acting on the segment.

FIG. 3 is a part of a catheter according to another embodiment of the present invention, illustrating an alternative view of a relationship between the shape of the catheter and its function.

FIG. 4 is a cross section view of a catheter according to another embodiment of the present invention.

FIG. 5 is an expanded view of a segment of the catheter shown in FIG. 4.

### DETAILED DESCRIPTION

Embodiments of the invention relate to catheters having unique profiles to prevent retention failure. Catheters of the invention, which may be referred to as “self-retaining” catheters, have unique profiles that interact with the compression or peristaltic actions of body cavities to enhance the retention of these catheters. Catheters of the invention may be used in any applications where conventional catheters are usede.

Catheters are inserted into a body cavity, such as urethra or blood vessel. The secretion (urination) action of urethra or the peristaltic movements (pulsations) of blood vessels may act to expel the catheters. Catheters of the invention include unique shapes such that these otherwise undesirable forces from the body cavities are harnessed to help retain the catheters in place.

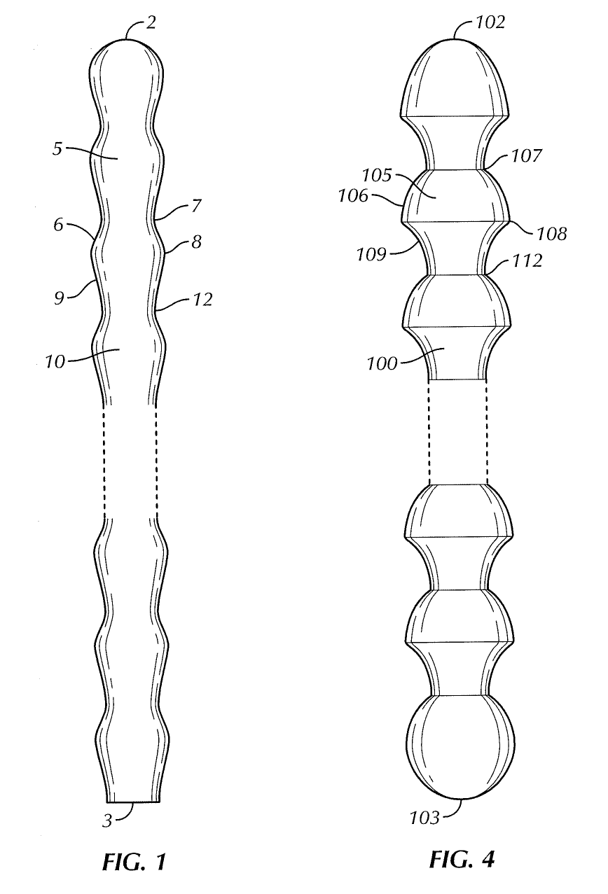

FIG. 1 shows a cross sectional view of a catheter in accordance with one embodiment of the present invention. As shown in FIG. 1, a catheter 10 includes a first end (front end) 2, a second end (back end) 3, and a body, comprising a plurality of segments 5, disposed between the first end 2 and the second end 3. The “first end” as used herein defines the insertion end (front end) of a catheter. Each segment 5 has a first tapered surface 6 with a diameter increasing from a first edge 7 to a tapered surface edge (a ridge) 8 and a second tapered surface 9 with a diameter decreasing from said ridge 8 to a second edge 12.

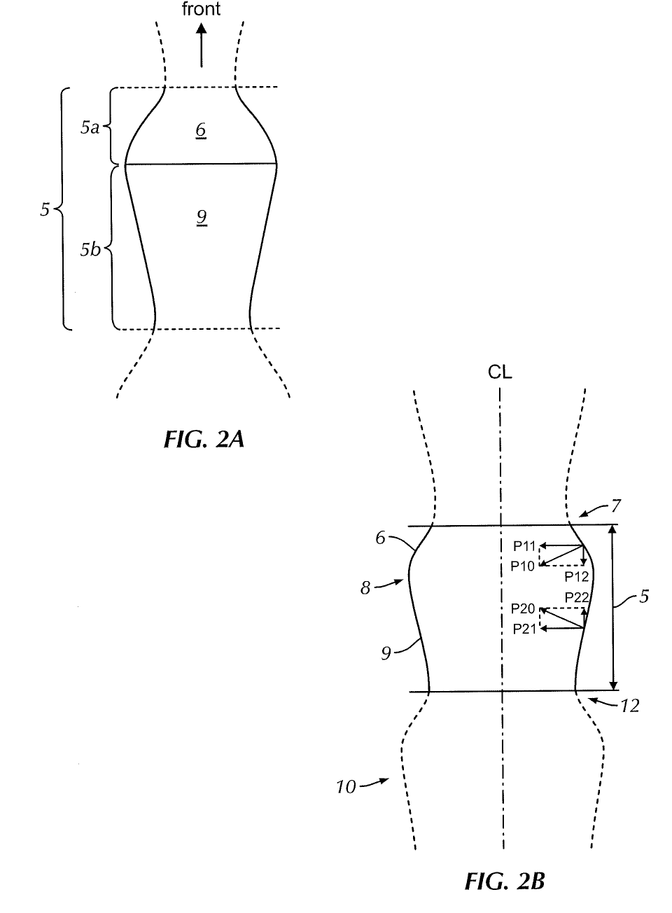

As shown in FIG. 1, the catheter 10 comprises a plurality of segments 5, which has a unique shape designed for enhanced retention of the catheter 10. FIG. 2A shows an expanded view of a segment 5 illustrating the unique structural features. As shown in FIG. 2A, the segment 5 comprises two sections 5a and 5b—the first section 5a is closer to the first end (or front end) 2 and has a first tapered surface 6, while the second section 5b is closer to the second end (or back end) 3 and has a second tapered surface 9. The two sections 5a and 5b join at a ridge 8, and extend to first edge 7 and second edge 12, respectively.

In this example, the second section 5b is longer than the first section 5a. Therefore, the surface area of the second tapered surface 9 is greater than the surface area of the first tapered surface 6. When the catheter 10 is inserted in a body cavity (e.g., urethra), the first and second tapered surfaces (6, 9) of each segment 5 will come into direct contact with and receive contact pressure from the inner surface of the body cavity. The contact pressure may be static or pulsating (e.g., in peristaltic actions of blood vessels). These contact pressures (forces) acting on the first tapered surface 6 and the second tapered surface 9 would affect the retention of the catheter 10 in the body cavity.

As shown in FIG. 2B, contact pressure P10 acting on the first tapered surface 6 works in a direction perpendicular to (i.e., normal to) the first tapered surface 6, and the pressure force may be separated into two force components, i.e., a first force P11 working in a direction perpendicular to the center line CL and a second force P12 working in a direction parallel with the center line CL. A sum of all first force P11 around the circumference at the same axial location will cancel out to produce a net force of zero, while a sum of all second force P12 around the same circumference will add up to a non-zero force that will act to push the catheter 10 out of urethra.

Similarly, contact pressure P20 acting on the second tapered surface 9 works in a direction perpendicular to (i.e., normal to) the second tapered surface 9, and the pressure force may be separated into two force components, i.e., a first force P21 working in a direction perpendicular to the center line CL and a second force P22 working in a direction parallel with the center line CL. A sum of all first force P21 around a circumference at the same axial location will cancel out to produce a zero force, while a sum of all second force P22 around the same circumference will add up to a non-zero force that will act to push the catheter 10 deeper into the urethra.

As shown in FIG. 2B, the sum of P12 forces act to push the catheter out, while the sum of P22 forces act to push the catheter in—i.e., they work in the opposite directions. In accordance with embodiments of the invention, because the second tapered surfaces 9 is larger than the first tapered surface 6, the sum of all P22 forces will be greater than the sum of all P12 forces. Therefore, the force pushing the catheter in would be greater than the force pushing it out. Accordingly, a catheter of the invention is less likely to fall out.

In addition, the friction force between the outside surface of the catheter and the inner surface of the body cavity (e.g., urethra) would also restrict the movement of a catheter, thereby helping to retain the catheter in place. By having one or more segments, each having a first and a second tapered surfaces, a catheter in accordance with embodiments of the invention necessarily has an increased overall surface, as compared with a straight, cylindrical catheter. Therefore, a catheter of the invention would also have a greater friction force, which would also help to keep the catheter in place.

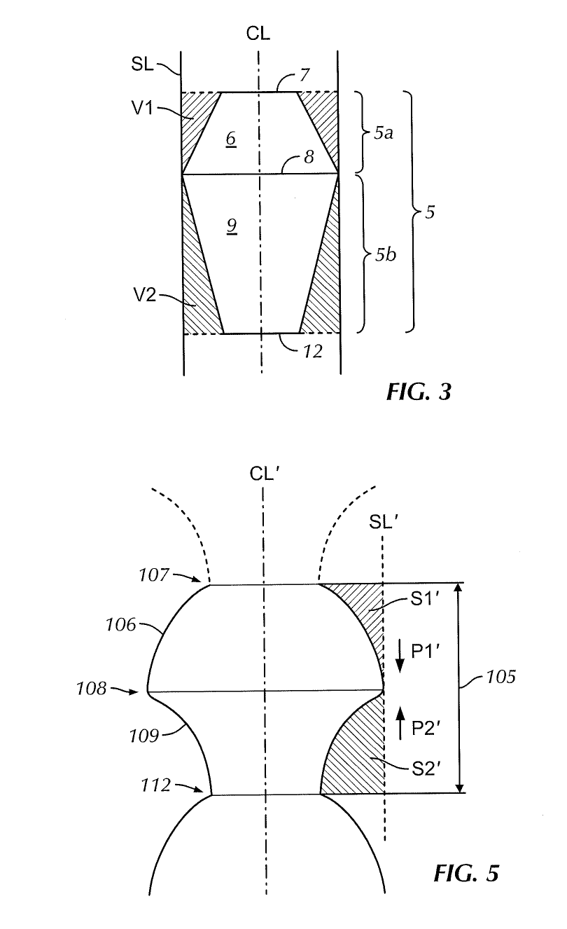

The embodiment shown in FIG. 1 has a plurality of segments 5, in which the first tapered surfaces 6 and the second tapered surfaces 9 join smoothly at ridge 8, at the first edges 7, and at the second edges 12. That is, the changes in diameters in the first section 5a and in the second section 5b occur gradually. Therefore, a cross section view of the catheter 10 shown in FIG. 1 has a curved first tapered surface 6 and a curved second tapered surface 9. The first tapered surface 6 and the second tapered surface 9 appear to have convex profiles in this example. However, embodiments of the invention are not limited to any particular shapes or profiles. Some embodiments of the invention may have the first tapered surface 6 and/or the second tapered surface 9 in concave profiles. Furthermore, in accordance with some embodiments of the invention, the first tapered surface 6 and/or the second tapered surface 9 may have a substantially straight (linear) profile, as illustrated in the embodiment shown in FIG. 3. In addition, a catheter of the invention may have segments comprising a combination (mix-and-match) of profiles (convex, concave, and/or straight) in some or all of the segments. Furthermore, a catheter of the invention may also include one or more sections that are straight cylinders as in a conventional catheter. In other words, catheters of the invention may comprise one or more segments described herein and the remaining parts of the catheters may be similar to a conventional catheter.

A catheter is to be inserted into a body cavity. As noted above, peristaltic movement in the body cavity may act to expel a catheter of a cylindrical tube. Thus, in some prior art approaches, external stop means (e.g., anchors) have been used to prevent retention failure of catheters. In accordance with embodiments of the invention, no external stop means is needed. Instead, catheters of the invention are designed with unique geometric shapes to harness these pulsating forces.

As illustrated in FIG. 2B, the pressure and peristaltic movement of the body cavity acting on the first tapered surface 6 may generate a thrust pushing the catheter 10 toward the second end 3, while the pressure of the body cavity acting on the second tapered surface 9 may generate a thrust pushing the catheter 10 towards the first end 2. By having a total surface area of the second tapered surfaces 9 greater than the total surface area of the first tapered surfaces 6, a catheter of the invention will have a net thrust pushing on the catheter towards the first end 2. Thus, the otherwise adverse effects of the peristaltic motions of the body cavity are actually used to help retain catheters of the invention in the body cavities.

As illustrated in FIG. 2B, the “self-retaining” feature of a catheter of the invention can be achieved if the forces acting on the second tapered surfaces 9 is greater than the forces acting of the first tapered surfaces 6. While the embodiment shown in FIG. 1 achieves this by having the second section 5b (see FIG. 2A) longer than the first section (or the area of the second tapered surface 9 greater than the area of the first tapered surface 6), other configurations of the segments are possible to achieve the same aim. This will be described in detail in a later section (see for example, FIGS. 4 and 5).

FIG. 3 shows a cross sectional view of a segment in accordance with one embodiment of the invention, illustrating another way to view the relationships between the shape of the catheter and its functions. In FIG. 3, an imaginary cylinder SL is shown circumscribing the segment 5 at the largest diameter location (i.e., at ridge 8). As used in this description, an imaginary cylinder circumscribing a segment is one circumscribing and touching the outer surface of the ridge 8. The central axis of the imaginary cylinder SL is aligned with the center line (or central axis) CL of the segment 5. A first volume V1 enclosed between the first tapered surface 6 and the imaginary cylinder sheath SL in the first section 5a is smaller than a second volume V2 enclosed between the second tapered surface 9 and the imaginary cylinder sheath SL in the second section 5b.

The diagram in FIG. 3 illustrates one criterion for determining whether a shape design of a segment 5 will have the desired feature of the invention. If the second volume V2 is larger than the first volume V1, then the forces acting on the second tapered surface 9 would be greater than the forces acting on the first tapered surface 6. As a result, the forces keeping the catheter in would be greater than the forces that would push the catheter out. Therefore, as long as one can achieve V2>V1, one would have a self-retaining catheter.

There are several ways to meet the V2>V1 requirement (or the ratio V2/V1>1). For example, the embodiment shown in FIG. 1 has a second section 5b longer than the first section 5a, which would result in V2>V1. Another way of achieving the V2>V1 relationship is illustrated in FIGS. 4 and 5. One skilled in the art would appreciate that these are examples only and are not intended to limit the scope of the invention and the other modifications and variations are possible to fulfill the V2>V1 relationship without departing from the scope of the invention.

In sum, the present inventor has found that if the shape of a segment 5 of a catheter is formed such that the ratio, V2/V1, is greater than 1, then the catheter may be stably maintained in the body cavity, and that the larger the ratio (V2/V1) is, the greater retention force a catheter would have.

As noted above, a catheter of the invention may comprise one or more segments having unique shapes to facilitate retention of the catheter. Various shapes of the segments may be adopted. For example, FIG. 4 shows a cross sectional view of a catheter according to another embodiment of the present invention. FIG. 5 shows an expanded view of a segment of the catheter shown in FIG. 4. FIG. 5 also describes an alternative parameter for determining whether a catheter would be self-retaining (this alternative parameter may be used instead of the V1 and V2 parameters discussed with reference to FIG. 3).

As shown in FIGS. 4 and 5, a catheter 100 includes a first end 102, a second end 103, and a plurality of segments 105 between the first end 102 and the second end 103. Each segment 105 has a first tapered surface 6 with a diameter increasing from a first edge 107 to a ridge 108 and a second tapered surface 9 with a diameter decreasing from said ridge 108 to a second edge 112. Note that the first tapered surface 106 has a convex profile in this cross sectional view, while that of the second tapered surface 109 has a concave profile.

The embodiment shown in FIGS. 4-5 may be viewed as having a first shaded area S1′ (which is proportional to the first volume V1 shown in FIG. 3) between the first tapered surface 106, and a hypothetical line SL′ (representing a line on the surface of the imaginary cylinder sheath SL shown in FIG. 3) parallel to the center line CL′ and passing the ridge 108, and a second shaded area S2′ (which is proportional to the second volume V2 shown in FIG. 3) between the second tapered surface 109 and the hypothetical line SL′. In this embodiment, the shape of the catheter is designed such that the shaded area S1′ is smaller than the shaded area S2′. That is, S2/S1>1 (which is equivalent to V2/V1>1). With this configuration (i.e., S2/S1>1), the net force acting on the segment would help to retain the catheter in place.

A catheter of the invention may be used in any situations where a catheter is needed. One exemplary use is in a urinary tract to drain and collect urine from a bladder. For example, such a catheter may be used for an elderly man with dementia or a patient bedridden for a medical treatment, etc. In another example, a catheter of the invention may be used in a blood vessel (e.g., for infusion of a medicine) or in a digestive tract.

The dimensions of such catheters may be sized according to the applications. For example, in the urinary catheter embodiments, the maximum diameter of the catheter (which may be the diameter of the ridge 8; see FIG. 1) may be about 8 mm. In addition, the size and shape of each segment may be varied. For example, a length of each segment 5 for a catheter may be equal to, larger than, or smaller than the diameter of the ridge 8. In accordance with some embodiments of the invention, a length of the segment 5 for a urinary catheter, for example, may be approximately 2 times or approximately 1.5 times the diameter of the ridge 8. In accordance with some embodiments of the invention, a catheter for use in a urethra, for example, may have a segment of 4 mm to 8 mm long.

Other embodiments of a self-retaining catheter according to embodiments of the invention for uses with other tubular cavities of the human body, (e.g., vascular system or the digestive system) will be accordingly sized. Therefore, the diameter of a self-retaining catheter of the invention can vary widely, for example, from less than 1 mm to as large as 25 mm or more, depending on the body cavity.

Embodiments of the invention may have one or more of the following advantages. Catheters of the invention have unique shapes that can harness the forces from a body cavity (e.g., a urethra or a blood vessel) and use that force to help retain the catheters in place. Catheters of the invention would be easy to manufacture and would be less intrusive because they do not include external anchor or stop means on the outside surfaces of the catheters. These catheters would be more comfortable for the users.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A catheter for use in a body cavity, comprising:
a first end;
a second end; and
a body, which comprises one or more segments, disposed between said first end and said second end,
wherein at least one of the one or more segments has a first section and a second section,
wherein the first section has a first tapered surface with a diameter increasing from a first edge to a ridge and a second section has a second tapered surface with a diameter decreasing from the ridge to a second edge,
wherein a first volume enclosed between said first tapered surface in the first section and an imaginary cylinder circumscribing the ridge is smaller than a second volume enclosed between the second tapered surface in the second section and the imaginary cylinder.

2. The catheter of claim 1, wherein the second section is longer than the first section.

3. The catheter of claim 1, wherein a length of the segment has is approximately 2 times a diameter at the ridge.

4. The catheter of claim 1, wherein a diameter at the ridge is about 8 mm.

5. The catheter of claim 1, wherein a profile of the first section and a profile of the second section are both convex.

6. The catheter of claim 1, wherein a profile of the first section is convex and a profile of the second section is concave.
